(19) United States
(12) Patent Application Publication
(10) Pub. No.: US 2025/0009499 A1
(43) Pub. Date: Jan. 9, 2025

(54) DEVICES AND METHODS FOR FORMING PROSTHETIC VALVE LEAFLETS

(71) Applicant: EDWARDS LIFESCIENCES CORPORATION, Irvine, CA (US)

(72) Inventor: Evan T. Schwartz, Huntington Beach, CA (US)

(21) Appl. No.: 18/887,376

(22) Filed: Sep. 17, 2024

Related U.S. Application Data
(63) Continuation of application No. PCT/US2023/017683, filed on Apr. 6, 2023.
(60) Provisional application No. 63/362,651, filed on Apr. 7, 2022.

(51) Int. Cl. A61F 2/24; A61F 2/00

(52) U.S. Cl. CPC A61F 2/2415; A61F 2/0095

(57) ABSTRACT

A packaging assembly for storing a prosthetic heart valve can comprise a valve tray comprising an annular rim and a cavity extending away from the annular rim, wherein the cavity is configured to accept the prosthetic heart valve. The packaging assembly can further comprise a lid configured to cover the annular rim. The packaging assembly can further comprise a leaflet biasing element disposed on a central portion of the lid. The leaflet biasing element can comprise an extrusion with a paraboloid shape that can extend from a lower surface of the lid towards the valve tray. The leaflet biasing element can be configured to extend at least partially into an inflow end portion of the prosthetic heart valve and secure a plurality of leaflets of the prosthetic heart valve in a desired configuration when the prosthetic heart valve is inserted in the cavity and the lid is closed over the annular rim.

### CROSS REFERENCE TO RELATED APPLICATION

This application is a continuation of PCT Patent Application No. PCT/US2023/017683, filed on Apr. 6, 2023, which claims the benefit of U.S. Provisional Application No. 63/362,651, filed Apr. 7, 2022, each of these applications being incorporated by reference herein in its entirety.

### FIELD

The present disclosure relates to devices and methods for forming leaflets of a prosthetic heart valve in a desired configuration.

### BACKGROUND

The human heart can suffer from various valvular diseases. These valvular diseases can result in significant malfunctioning of the heart and ultimately require repair of a native valve of the heart or replacement of the native valve with an artificial valve. In some heart valve replacement procedures, the native valve can be replaced with a prosthetic heart valve, which can utilize fluid-occluding leaflets formed from biologically derived tissues. Some examples of the prosthetic heart valve can comprise a whole porcine valve and a plurality of leaflets made from bovine pericardium and stitched together to form a tri-leaflet valve.

When implanted at a native valve site, the prosthetic heart valve can regulate blood flow driven by a pressure gradient across the prosthetic heart valve. For example, the prosthetic heart valve, when implanted at a mitral or a tricuspid valve site, can permit blood flow from the heart's atrial chambers by opening the plurality of leaflets during ventricular diastole and can inhibit blood flow into the atrial chambers by closing the plurality of leaflets during ventricular systole. Similarly, a prosthetic heart valve implanted at an aortic or a pulmonary valve site can permit blood flow from the heart's ventricular chambers by opening the plurality of leaflets during ventricular systole and inhibit blood flow into the ventricular chambers by closing the plurality of leaflets during ventricular diastole.

### SUMMARY

The present application discloses a prosthetic heart valve with a plurality of leaflets in accordance with certain examples of the invention that can solve one or more deficiencies in the prior art. In particular, the prosthetic heart valve can comprise leaflets biased or shape-set to a desired configuration that better facilitates full actuation of the leaflets between an open configuration and a closed configuration. Furthermore, the present application discloses a packaging assembly that can be configured to bias the plurality of leaflets in the desired configuration. Furthermore, the present application discloses a method of shape-setting the plurality of leaflets in the desired configuration.

In a representative example, a packaging assembly for storing a prosthetic heart valve can comprise a valve tray comprising an annular rim and a cavity extending away from the annular rim, wherein the cavity is configured to accept the prosthetic heart valve. The packaging assembly can further comprise a lid configured to cover the annular rim. The packaging assembly can further comprise a leaflet biasing element disposed on a central portion of the lid. The leaflet biasing element can comprise an extrusion with a paraboloid shape. The leaflet biasing element can extend from a lower surface of the lid towards the valve tray. The leaflet biasing element can be configured to extend at least partially into an inflow end portion of the prosthetic heart valve and secure a plurality of leaflets of the prosthetic heart valve in a desired configuration when the prosthetic heart valve is inserted in the cavity and the lid is closed over the annular rim.

In another representative example, a packaging assembly for storing a prosthetic heart valve can comprise a valve tray configured to accept the prosthetic heart valve and a leaflet biasing element configured to protrude into the valve tray, wherein the leaflet biasing element is configured to secure a plurality of leaflets of the prosthetic heart valve in a desired configuration.

In another representative example, a packaging assembly for storing a prosthetic heart valve can comprise a valve tray and a lid. The valve tray can comprise an annular rim, a cavity extending in a direction away from the annular rim, wherein the cavity is configured to accept the prosthetic heart valve, and a leaflet biasing element disposed on a central portion of a bottom surface of the cavity. The leaflet biasing element can extend from the bottom surface towards the annular rim and can be configured to secure a plurality of leaflets of the prosthetic heart valve in a desired configuration when the prosthetic heart valve is seated in the cavity. The lid can be configured to cover the annular rim.

In another representative example, a packaging assembly for storing a prosthetic heart valve can comprise a first portion and a second portion. The first portion can comprise an annular rim and a cavity extending in a direction away from the annular rim, wherein the cavity is configured to accept the prosthetic heart valve. The second portion can comprise a lid configured to cover the annular rim and a leaflet biasing element extending from a central portion of the lid towards the lower half. The leaflet biasing element can be configured to secure a plurality of leaflets of the prosthetic heart valve in a desired configuration when the prosthetic heart valve is inserted into the cavity and the second portion is closed over the first portion.

In another representative example, a packaging assembly for storing a prosthetic heart valve can comprise an inner packaging member, an outer packaging member configured to accept the inner packaging member, and a gas-permeable lid configured to seal the outer packaging member when the inner packaging member is inserted into the outer packaging member. The inner packaging member can comprise a first portion comprising a circular aperture and a cavity extending downwardly from the circular aperture, wherein the cavity is configured to accept the prosthetic heart valve. The inner packaging member can further include a second portion comprising a lid configured to cover the circular aperture and a paraboloid-shaped leaflet biasing element extending into the cavity. The leaflet biasing element can be configured to secure a plurality of leaflets into a desired configuration; and a living hinge coupling the first portion to the second portion.

In another representative example, a packaging assembly for storing a prosthetic heart valve can comprise an inner packaging member, a storage tray, and a gas-permeable lid. The inner packaging member can comprise a first portion and a second portion, wherein the first portion comprises an annular rim surrounded by a circular aperture, a lower ledge defined by and disposed beneath the circular aperture, a first cavity extending downwardly from the lower ledge and configured to accept the prosthetic heart valve, a plurality of radially-extending lower gas channels disposed on the annular rim, and a lower finger tab radially extending from the annular rim. The second portion can comprise a lid configured to cover the circular aperture and a leaflet biasing element extending from a central portion of the lid towards the first portion, wherein the leaflet biasing element can extend at least partially into the first cavity to secure a plurality of leaflets of the prosthetic heart valve in a desired configuration. The second portion can further comprise a plurality of radially-extending upper gas channels disposed on the lid, wherein each of the upper gas channels can be configured to align with a respective one of the lower gas channels, and an upper finger tab extending from the lid and aligned with the lower finger tab. The storage tray can comprise an open end and a second cavity extending from the open end, wherein the second cavity is configured to accept the clamshell. The gas-permeable lid can be configured to fit over the open end of the storage tray, wherein the gas-permeable lid can be configured to permit gas to enter the valve second cavity but prevent ingress of microorganisms.

In a representative example, a method of biasing a plurality of leaflets of a prosthetic heart valve in a desired configuration can comprise dehydrating the prosthetic heart valve, securing the plurality of leaflets in the desired configuration, wherein the desired configuration is configured to reduce retrograde flow through the prosthetic heart valve, and sterilizing the prosthetic heart valve.

In another representative example, a method of shape-setting a plurality of leaflets of a prosthetic heart valve to a desired configuration can comprise dehydrating the plurality of leaflets, inserting the prosthetic heart valve into a packaging member, closing the lid such that the leaflet biasing element engages the plurality of leaflets, and sterilizing the prosthetic heart valve. The packaging member can comprise a cavity configured to accept the prosthetic heart valve, a lid configured to seal the cavity, and a leaflet biasing element disposed on a central portion of a bottom surface of the lid, wherein the leaflet biasing element is configured to extend into the cavity and is configured to secure the plurality of leaflets in the desired configuration.

In another representative example, a method of shape-setting a prosthetic heart valve can comprise dehydrating a plurality of leaflets of the prosthetic heart valve by treating the prosthetic heart valve with a non-aqueous treatment solution comprising a glycerol and an alcohol, removing a portion of the non-aqueous treatment solution from the plurality of leaflets, inserting a leaflet biasing element at least partially into a central flow orifice formed by the prosthetic heart valve, wherein the leaflet biasing element is configured to secure the plurality of leaflets towards a circumferential portion of the central flow orifice, and sterilizing the prosthetic heart valve by exposing the prosthetic heart valve to an ethylene oxide gas.

In another representative example, a method of fabricating a prosthetic heart valve—wherein the prosthetic heart valve comprises a plurality of leaflets formed from bovine pericardium—can comprise immersing at least the plurality of leaflets in a non-aqueous treatment solution comprising a glycerol and a C₁-C₃ alcohol selected from the group consisting of methanol, ethanol, n-propanol, 2-propanol and mixtures thereof, wherein the glycerol is 60-95% by volume. The method can further comprise removing a portion of the non-aqueous treatment solution from the plurality of leaflets, inserting the prosthetic heart valve into a gas-permeable packaging member, wherein the gas-permeable packaging member is configured to secure the plurality of leaflets in a desired configuration, and sterilizing the prosthetic heart valve by exposing the gas-permeable packaging member to an ethylene oxide gas.

In one representative example, a prosthetic heart valve can comprise an annular frame extending from an inflow end portion towards an outflow end portion of the prosthetic heart valve, a plurality of commissure supports circumferentially disposed around the annular frame and projecting towards the outflow end portion, and a plurality of leaflets secured to the plurality of commissure supports, wherein the plurality of leaflets can be formed from bovine pericardium tissue, Each of the plurality of leaflets can comprise a free edge portion between two lateral portions. Each of the plurality of leaflets can be biased to an open configuration in which the free edge portion generally follows the annular frame in a circumferential direction, the two lateral portions are secured to adjacent ones of the plurality of commissure supports, and the prosthetic heart valve is configured to be stored in a dry state.

In another representative example, a prosthetic heart valve can comprise an annular frame extending from an inflow end portion towards an outflow end portion of the prosthetic heart valve, a plurality of commissure supports circumferentially disposed around the annular frame and projecting towards the outflow end portion, and a plurality of leaflets secured to the plurality of commissure supports, wherein the plurality of leaflets are biased to a desired configuration, wherein the prosthetic heart valve is configured to be stored in a dry state.

In another representative example, a prosthetic heart valve can comprise an annular frame extending from an inflow end portion of the prosthetic heart valve towards an outflow end portion of the prosthetic heart valve, wherein the annular frame forms a central flow orifice extending from the inflow end portion to the outflow end portion and through the annular frame. The prosthetic heart valve can further comprise a plurality of commissure supports circumferentially disposed around the annular frame and projecting towards the outflow end portion and a plurality of leaflets secured to the plurality of commissure supports, wherein the plurality of leaflets are dehydrated and sterilized such that the prosthetic heart valve is configured to be stored in a dry environment, and wherein the plurality of leaflets are shape-set to a desired configuration after the plurality of leaflets are dehydrated and sterilized.

In another representative example, a prosthetic heart valve can comprise an annular frame extending from an inflow end portion of the prosthetic heart valve towards an outflow end portion of the prosthetic heart valve, wherein the annular frame forms a central flow orifice extending from the inflow end portion to the outflow end portion and through the annular frame, a plurality of commissure supports circumferentially disposed around the annular frame and projecting towards the outflow end portion, and a plurality of leaflets formed from bovine pericardium tissue, wherein each of the plurality of leaflets comprises a free edge portion between two lateral portions, the two lateral portions are secured to adjacent ones of the plurality of commissure supports, each of the plurality of leaflets is biased to a desired configuration, and the prosthetic heart valve is configured to be stored in a dry state.

### BRIEF DESCRIPTION OF THE DRAWINGS

FIG. 1 is a perspective view of a prosthetic heart valve, according to one example.

FIG. 2A is an outflow view of a prosthetic heart valve with one leaflet in a closed configuration, according to one example.

FIG. 2B is an outflow view of a prosthetic heart valve in an open configuration, according to another example.

FIG. 2C is an outflow view of a prosthetic heart valve in a closed configuration, according to another example.

FIG. 3 is a perspective view of a clamshell member for storing a prosthetic heart valve, according to one example.

FIG. 4 is a side view of a clamshell member for storing a prosthetic heart valve, according to one example.

FIG. 5 is an exploded perspective view of a packaging assembly for storing a prosthetic heart valve, according to one example.

FIG. 6 is a perspective view of a prosthetic heart valve, according to another example.

FIG. 7 is a perspective view of a surgical valve, according to one example.

FIG. 8 is a perspective view of a clamshell member for storing a prosthetic heart valve, according to another example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled,” “connected,” and “joined” generally mean physically, mechanically, chemically, magnetically, and/or electrically coupled or linked and does not exclude the presence of intermediate elements between the coupled or associated items absent specific contrary language.

As used in this application and in the claims, the term “shape-setting” can generally refer to a process of plastically deforming a material from a first configuration to a second configuration. The shape-setting process can result in the material becoming biased to the second configuration. In some examples, shape-setting can occur as a result of a physical stimulus, a chemical reaction, or a combination thereof.

In some examples, the shape-setting process can further impart shape-memory properties onto the material. As used in this application and in the claims, the term “shape-memory” can generally refer to the tendency of a material to elastically deform or return from a third configuration to the second configuration after the material is deformed from the second configuration to the third configuration. In some examples, the shape-memory property can be activated by an external stimulus, such as a temperature change, chemical reaction, or physical force.

As used in this application and in the claims, the term “inflow” can generally refer to a position, direction, or portion of the prosthetic heart valve that is closer an inlet into which blood flow enters the prosthetic heart valve. As used in this application and in the claims, the term “outflow” can generally refer to a position, direction, or portion of a prosthetic heart valve that is closer an outlet from which blood flow exits the prosthetic heart valve. As used in this application and in the claims, the terms “longitudinal” and “axial” can generally refer to an axis extending in the inflow and outflow directions, unless otherwise expressly defined.

As used in this application and in the claims, the terms “dry prosthetic heart valve” or “dry bioprosthetic heart valve” can generally refer to a prosthetic heart valve made of dehydrated bioprosthetic tissue. Dehydrating the bioprosthetic tissue beneficially allows the prosthetic heart valve to be stored in a non-fluid environment prior to surgical implantation. Dry storage can beneficially eliminate the need to store the prosthetic heart valve in formaldehyde, glutaraldehyde, or other potentially toxic storage solutions that can detrimentally calcify the bioprosthetic tissue. Furthermore, dry storage can beneficially eliminate the need to rinse the prosthetic heart valve of the storage solutions prior to surgical implantation.

### Overview of the Disclosed Technology

A prosthetic heart valve can be implanted in a patient to replace a diseased or non-functioning native heart valve. The prosthetic heart valve can comprise a frame and a plurality of leaflets. The frame can comprise a porcine valve, while the plurality of leaflets can comprise a bioprosthetic tissue such as bovine pericardium tissue. The plurality of leaflets can actuate between an open configuration and a closed configuration. Examples of the prosthetic heart valve can be found in U.S. Patent Application Publication No. 2018/0206982, filed on Jul. 26, 2018, which is herein incorporated by reference. Other examples of prosthetic heart valves can include such valves as those which are part of the Edwards SAPIEN 3 and SAPIEN 3 Ultra Transcatheter Heart Valve Systems of Edwards Lifesciences in Irvine, California, and any other transcatheter prosthetic heart valves that are designed to be implanted in the patient.

The opening and closing of the plurality of leaflets is driven by a pressure gradient across the prosthetic heart valve. However, a high pressure gradient can cause the prosthetic heart valve to fail by preventing the plurality of leaflets from fully opening or closing. For example, if the pressure in the left ventricular chamber is too high relative to the pressure in the aorta, the plurality of leaflets of an aortic valve might stick in the open configuration and not fully close during ventricular systole. Similarly, if the pressure in the left ventricular chamber is too high relative to the pressure in the left atrial chamber, the plurality of leaflets of a mitral valve might stick in the closed configuration and not fully open during ventricular diastole. In some examples, the plurality of leaflets may fail to open or close because they lack chordae tendineae connecting them to the papillary muscles, which typically help actuate leaflets of the native valve.

If the plurality of leaflets do not fully open or close, the pressure gradient across the prosthetic valve can result in aortic valve stenosis or mitral valve regurgitation, respectively. Aortic valve stenosis can detrimentally occlude blood flow, while mitral valve regurgitation can detrimentally lead to retrograde blood flow. Both aortic valve stenosis and mitral valve regurgitation can lead to heart failure or even death. Thus, there is a need to ensure that the plurality of leaflets can fully actuate between the open configuration and the closed configuration.

In some examples, the prosthetic heart valve can be a dry prosthetic heart valve, in which the bioprosthetic tissue comprising the prosthetic heart valve is dehydrated. In one example, the prosthetic heart valve can be dehydrated by treating the plurality of leaflets with a non-aqueous treatment solution comprising a polyhydric alcohol and a C₁-C₃ alcohol, after which a portion of the treatment solution can be removed from the solution-treated biological tissue. In some of these examples, the plurality of leaflets can be pretreated with an aldehyde capping agent prior to dehydration and sterilization to reduce in vivo calcification of the plurality of leaflets. Further exemplary bioprosthetic tissue dehydration processes can be found in U.S. Patent Application Publication No. 2009/0164005, filed on Dec. 18, 2008, and in U.S. Patent Application Publication No. 2008/0102439, filed on Oct. 23, 2007, which are both herein incorporated by reference. Other exemplary bioprosthetic tissue dehydration processes can include the RESILIA® bioprosthetic tissue treatment process from Edwards Lifesciences in Irvine, California. However, other suitable bioprosthetic tissue treatment processes can be used to dehydrate the bioprosthetic tissue.

After the prosthetic heart valve has been dehydrated, it can be packaged in a packaging assembly to prevent damage to the prosthetic heart valve prior to surgical implantation. Some exemplary packaging assemblies for prosthetic heart valves can be found in U.S. Patent Publication No. 2011/0214398, filed on Mar. 2, 2011, and U.S. Patent Publication No. 2012/0158128, filed on Dec. 13, 2011, which are both incorporated herein by reference. However, other suitable packaging assemblies can be used.

After the prosthetic heart valve has been packaged, the prosthetic heart valve can be sterilized to prevent microbial and/or fungal growth. In some examples, the prosthetic heart valve can be sterilized by an exposure to a gaseous environment. For example, the prosthetic heart valve can be exposed to a gas comprising ethylene oxide and hydrochlorofluorocarbon. In other examples, the prosthetic heart valve can be sterilized by an exposure to ionizing radiation, including gamma and e-beam radiation.

In some examples, one or more of the plurality of leaflets of the prosthetic heart valve can shape-set or plastically deform during the dehydration, packaging, and/or sterilization processes. For example, the plurality of leaflets can shape-set when the prosthetic heart valve is dehydrated using the RESILIA® bioprosthetic tissue treatment process and subsequently sterilized using ethylene oxide gas. However, in other examples, shape-setting can occur as a result of different prosthetic heart valve fabrication processes.

A shape-set leaflet can become biased to a shape-set configuration. In some examples, this can mean that the shape-set leaflet plastically deforms to the shape-set configuration. In some examples, this can mean that the shape-set leaflet elastically deforms into a deformed configuration when subjected to an external force but reverts from the deformed configuration to the shape-set configuration when the external forces are removed. In some examples, shape-setting can stiffen the leaflet, requiring more force to elastically deform the shape-set leaflet from the shape-set configuration to the deformed configuration as compared to a force required to similarly deform a non-shape-set leaflet.

In some examples, the plurality of leaflets can become shape-set to the open configuration, the closed configuration, or an intermediate configuration between the open configuration and the closed configuration. The plurality of leaflets in the open configuration can open to permit blood flow through the prosthetic heart valve, while the plurality of leaflets in the closed configuration can close to occlude blood flow through the prosthetic heart valve. If the plurality of leaflets become shape-set to the closed configuration, the plurality of leaflets can require more force to open. Similarly, if the plurality of leaflets become shape-set to the open configuration, the plurality of leaflets can require more force to close. Thus, there is a need to prevent the plurality of leaflets from becoming biased to an undesirable configuration.

Thus, as used in this application and in the claims, the term “dry” can refer to a non-fluid environment with a water content no higher than the surrounding environment.

### Examples of the Disclosed Prosthetic Heart Valves

The prosthetic heart valves disclosed throughout this application and the claims can alternatively be referred to as dry bioprosthetic heart valves, dry prosthetic heart valves, bioprosthetic heart valves, prosthetic valves, artificial heart valves, or artificial valves. The prosthetic heart valves can be adapted to be implanted in a native annulus, including a mitral annulus, a pulmonary annulus, an aortic annulus, and/or a tricuspid annulus. The disclosed prosthetic heart valves also can be implanted within vessels communicating with the heart, including a pulmonary artery (for replacing the function of a diseased pulmonary valve), the superior vena cava or the inferior vena cava (for replacing the function of a diseased tricuspid valve) or various other veins, arteries and vessels of a patient. The disclosed prosthetic heart valves also can be implanted within a previously implanted prosthetic valve (which can be a prosthetic surgical valve or a prosthetic transcatheter heart valve) in a valve-in-valve procedure.

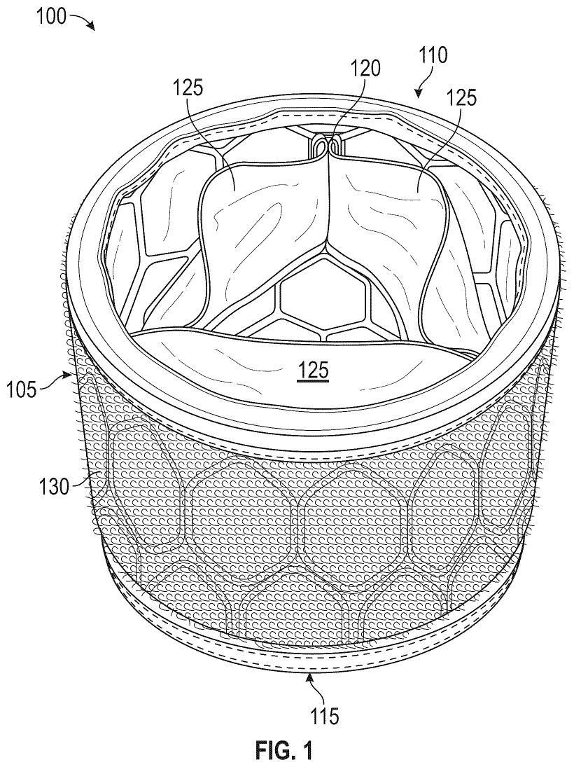

FIG. 1 illustrates a perspective view of a prosthetic heart valve 100, according to one example. The illustrated prosthetic heart valve 100 can be adapted to be implanted in the native mitral annulus. However, other examples of the prosthetic heart valve 100 can be implanted in the native aortic annulus, tricuspid annulus, pulmonary annulus, or other native valve sites. The prosthetic heart valve 100 can comprise a frame 105, a plurality of commissures 120, a plurality of leaflets 125, and an outer skirt 130. However, other examples of the prosthetic heart valve 100 can comprise additional or alternative components.

Examples of prosthetic heart valves include Edwards SAPIEN 3 and SAPIEN 3 Ultra Transcatheter Heart Valve System, and transcatheter prosthetic heart valves described for example in U.S. Patent Application Publication No. 2018/0206982, filed on Jul. 26, 2018. The illustrated prosthetic heart valve 100 can correspond to these and other models or examples of prosthetic heart valves.

The frame 105 can comprise an annular structure such as a cylinder. The circumference of the cylinder can form a central flow orifice through which blood can flow. The cylinder can be aligned such that the central flow orifice extends from an inflow end portion 115 at an inflow end of the frame 105 to an outflow end portion 110 at the outflow end of the frame 105. The frame 105 can be made of any suitable metal, plastic, tissue, or other material. In some examples, the frame 105 can comprise a porcine valve. In other examples, the frame 105 can comprise a metal wire or mesh frame. In some of these examples, the metal wire or mesh frame can be radially expandable or compressible.

The dry bioprosthetic valve 100 can further comprise the plurality of commissures 120 for securing the plurality of leaflets 125 to the frame 105. The plurality of commissures 120, which can also be referred to as commissure posts or commissure supports, can comprise a plurality of upstanding posts circumferentially distributed around the frame 105 and projecting towards the outflow end portion 110. In some examples, the plurality of commissures 120 can be extensions of the frame 105. The plurality of commissures 120 can be made of any suitable metal, polymer, plastic, bioprosthetic tissue, suturing material, or other material.

The dry bioprosthetic valve 100 can further comprise the plurality of leaflets 125, which can be configured to regulate blood flow through the dry bioprosthetic valve 100. In the example depicted in FIG. 1, the plurality of leaflets 125 can include three leaflets arranged to collapse in a tricuspid arrangement. However, other examples of the prosthetic heart valve 100 can include the three leaflets arranged to collapse in a different arrangement or can comprise a different number of leaflets.

Each of the plurality of leaflets 125 can include two lateral portions. Each lateral portion can define an edge that extends from the inflow end portion 115 towards the outflow end portion 110. The lateral portions can be aligned with and secured to the plurality of commissures 120. For example, each of the two lateral portions can be secured to an adjacent, corresponding one of the plurality of commissures 120.

Each of the plurality of leaflets 125 can further include an inflow edge portion (which can also be referred to as a cusp edge portion) extending between the two lateral portions on a portion of the leaflet closest to the inflow end portion 115. The inflow edge portions can define an undulating, curved scallop edge that generally follows or tracks portions of the frame 105 in a circumferential direction. The inflow edge portions of the plurality of leaflets 125 can be referred to as a “scallop line.”

Each of the plurality of leaflets 125 can further include a free edge portion on a portion of the leaflet between the two lateral portions and closest to the outflow end portion 110. The free edge portion of each of the plurality of leaflets 125 can extend inward towards the central flow orifice and coapt, or mate, with the free edge portions of the other leaflets when the plurality of leaflets 125 are in a closed configuration, so as to occlude blood flow through the central flow orifice. When the plurality of leaflets 125 are in an open configuration, the free edge portion of each of the plurality of leaflets 125 can generally follow portions of the frame 105 in the circumferential direction, so as not to occlude blood flow through the central flow orifice.

The plurality of leaflets 125 can be biased or shape-set. In some examples, like the example in FIG. 1, the plurality of leaflets 125 can be shape-set to the open configuration. Shape-setting the plurality of leaflets 125 in the open configuration can beneficially reduce the amount of force needed to actuate the plurality of leaflets 125 from the closed configuration to the open configuration, thereby improving the actuation of the plurality of leaflets 125 between the open and closed configurations during normal valve operation. In other examples, the plurality of leaflets 125 can be shape-set to the closed configuration or an intermediate configuration between the open configuration and the closed configuration.

In some examples, the plurality of leaflets 225 can be made from a bioprosthetic tissue, such as bovine pericardium. In these examples, the plurality of leaflets 225 can be formed by cutting the leaflets from a pericardial sac of a cow. However, other examples of the plurality of leaflets 225 can comprise any other suitable tissue or material. The bioprosthetic tissue can be treated or dehydrated such that the prosthetic heart valve 200 can be stored in a non-aqueous environment, i.e., not stored in a liquid preservative, prior to surgical implantation. Furthermore, the bioprosthetic tissue can be sterilized to prevent microbial or fungal growth.

In some examples, the prosthetic heart valve 100 can include one or more skirts mounted around the frame 105. For example, as shown in FIG. 1, the prosthetic heart valve 100 can include the outer skirt 130 mounted around an outer surface of the frame 105. The outer skirt 130 can function as a sealing member for the prosthetic heart valve 100 by sealing against the tissue of the native valve annulus and helping to reduce paravalvular leakage past the prosthetic heart valve 100.

In some cases, an inner skirt (not shown) can be mounted around an inner surface of the frame 105. The inner skirt can function as a sealing member to prevent or decrease perivalvular leakage, to anchor the plurality of leaflets 125 to the frame 105, and/or to protect the plurality of leaflets 125 against damage caused by contact with the frame 105 during crimping and during working cycles of the prosthetic heart valve 100. In some examples, inflow edge portions of the plurality of leaflets 125 can be sutured to the inner skirt generally along the scallop line. The inner skirt can in turn be sutured to the frame 105. In other examples, the plurality of leaflets 125 can be sutured directly to the frame 105.

The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valves can be found, for example, in U.S. Patent Publication No. 2020/0352711, which is incorporated herein by reference.

Finally, an identification tag (not pictured) can be secured to the frame 105 with a length of suture. The identification tag can provide a serial number representative of information regarding the type of heart valve and other particularities about its manufacture, such as the date.

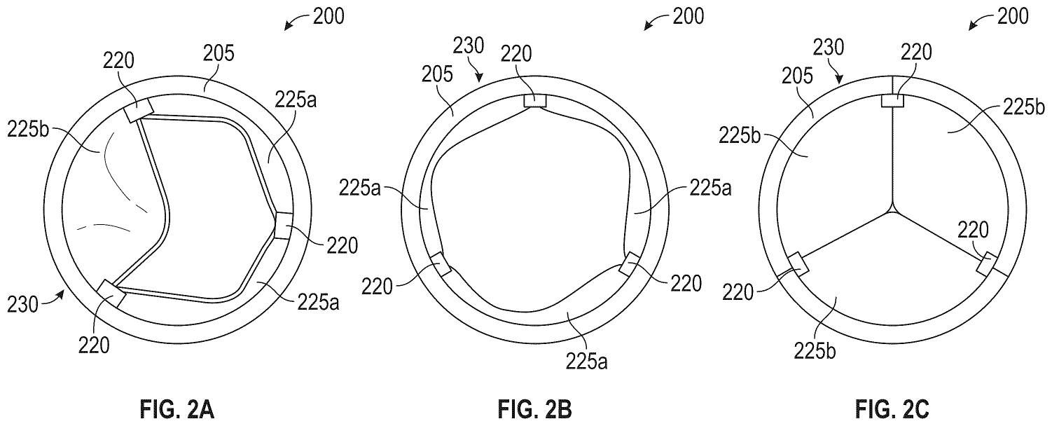

FIG. 2A depicts an example prosthetic heart valve 200 viewed from an outflow end of the prosthetic heart valve 200. The illustrated prosthetic heart valve 200 can comprise a frame 205, a plurality of commissures 220, two open leaflets 225a in the open configuration, one closed leaflet 225b shape-set to the closed configuration, and an outer skirt 230. However, other examples of the prosthetic heart valve 200 can comprise additional or alternative components.

The plurality of commissures 220 can be circumferentially arranged around the frame 205 and extend from the frame 205 towards the outflow end of the prosthetic heart valve 200. The open leaflets 225a and the closed leaflet 225b can be secured to the plurality of commissures 220.

The prosthetic heart valve 200 illustrated in FIG. 2A can be implanted at a native valve annulus of a heart surgery patient. During normal valve operation, the leaflets 225a, 225b of the prosthetic heart valve 200 can actuate between the open configuration and the closed configuration to regulate blood flow through the native valve annulus. However, the closed leaflet 225b can require more force to actuate into the open configuration if it is set-shape in the closed configuration. In the illustrated example, the prosthetic heart valve 200 can be subjected to sufficient force to actuate the open leaflets 225a, but not the closed leaflet 225b, to the open configuration. However, in other examples, both the open leaflets 225a and the closed leaflet 225b can actuate to the open configuration, but the closed leaflet 225b can simply require more force to actuate to the open configuration. Thus, there exists a need to facilitate the actuation of the closed leaflet 225b between the closed configuration and the open configuration.

FIG. 2B illustrates the prosthetic heart valve 200 from the outflow end portion of the prosthetic heart valve 200, according to another example. The prosthetic heart valve 200 can be configured for implantation in the mitral annulus. However, other examples of the prosthetic heart valve 200 can be implanted at other locations.

As illustrated in FIG. 2B, the open leaflets 225a can be shape-set or biased to the open configuration, in which the free edge of each of the open leaflets 225a generally follows the frame 205 in a circumferential direction. In some examples, such as the example illustrated in FIG. 2A, the free edge of each of the open leaflets 225a can form a generally bell-shaped curve, wherein the tip of the bell-shaped curve extends outwards towards the circumference of the frame 205 and each end portion of the bell-shaped curve terminates at one of the plurality of commissures 220.

In some examples, shape-setting or biasing the open leaflets 225a in the open configuration can improve the performance of the prosthetic heart valve 200 when the prosthetic heart valve 200 is implanted in the mitral annulus. Biasing or shape-setting the open leaflets 225a in the open configuration can help counteract the pressure gradient across the mitral annulus that tends to force a heart valve's leaflets into the closed configuration. Furthermore, shape-setting the open leaflets 225a in the open configuration can help compensate for the prosthetic heart valve's lack of chordae tendineae connecting the open leaflets 225a to the papillary muscles, which typically help actuate leaflets of the native valve.

FIG. 2C illustrates the prosthetic heart valve 200 from the outflow end portion of the prosthetic heart valve 200, according to another example. The prosthetic heart valve 200 can be configured for implantation in the aortic annulus. However, other examples of the prosthetic heart valve 200 can be implanted at other locations.

As illustrated in FIG. 2C, the closed leaflets 225b are shape-set or biased to the closed configuration, in which the free edge of each of the closed leaflets 225b extends inwards towards the central flow orifice at the center of the frame 205. In some examples, the free edges of the closed leaflets 225b extend towards each other such that a gap forms at a central portion of the central flow orifice. In other examples, the closed leaflets 225b extend into the central portion of the central flow orifice to substantially occlude any blood flow from passing through the central flow orifice.

In some examples, shape-setting or biasing the closed leaflets 225b in the closed configuration can improve the performance of the prosthetic heart valve 200 when the prosthetic heart valve 200 is implanted in the aortic annulus. Biasing or shape-setting the closed leaflets 225b in the closed configuration can help counteract the pressure gradient across the aortic annulus that tends to force a heart valve's leaflets into the open configuration. Furthermore, shape-setting the closed leaflets 225b in the closed configuration can help compensate for the prosthetic heart valve's lack of chordae tendineae connecting the closed leaflets 225b to the papillary muscles, which typically help actuate leaflets of the native valve.

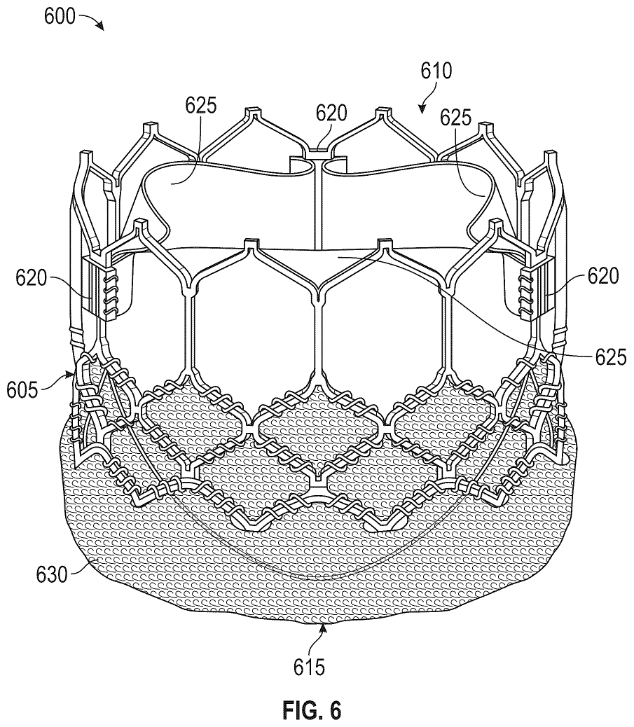

FIG. 6 depicts an example prosthetic heart valve 600, according to another example. The prosthetic heart valve 600 can comprise a frame 605, a plurality of commissures 620, a plurality of leaflets 625, and an inner skirt 630. However, other examples of the prosthetic heart valve 600 can comprise additional or alternative components.

Some examples of the prosthetic heart valve 600 can include the SAPIEN 3 transcatheter heart valve from Edwards Lifesciences of Irvine, California. However, other examples of the prosthetic heart valve 600 can include different models from different sources.

In the illustrated example, each of the plurality of leaflets 625 can comprise a free edge portion disposed towards an outflow end portion 610 of the prosthetic heart valve 600, wherein the free edge portion is adjacent two lateral portions. Each of the two lateral portions, which extend between an inflow end portion 615 and the outflow end portion 610, are secured to corresponding adjacent ones of the plurality of commissures 620 while the free edge portion remains unattached to the plurality of commissure posts 620 or the frame 605.

In the illustrated example, each of the plurality of leaflets 625 can be biased or shape-set to the open configuration, in which the free edge portion of each of the plurality of leaflets 625 generally follows or tracks the frame 605 in a circumferential direction. In some examples, each of the plurality of leaflets 625 can form a generally bell-shaped curve, wherein the tip of the bell-shaped curve extends outwards towards the circumference of the frame 605 and each end portion of the bell-shaped curve terminates at one of the plurality of commissures 620.

In some examples, in which the prosthetic heart valve 600 is implanted in a native mitral annulus, biasing or shape-setting the plurality of leaflets 625 in the open configuration can beneficially reduce the amount of force needed to open the plurality of leaflets 625, thereby facilitating the actuation of the plurality of leaflets 625 from the closed configuration to the open configuration.

However, in other examples, the prosthetic heart valve 600 can be implanted in an aortic annulus or any other suitable annulus in a patient's heart. If the prosthetic heart valve 600 is implanted in the aortic annulus, the plurality of leaflets 625 can be biased or shape-set to a closed configuration, in which the free edge portions of each of the plurality of leaflets 625 extend into a central flow orifice formed by the frame 605. The plurality of leaflets 625 in the closed configuration can occlude blood flow through the central flow orifice formed by the frame 605. In these examples, biasing or shape-setting the plurality of leaflets 625 in the closed configuration can help counteract the pressure gradient across the aortic annulus that tends to force the plurality of leaflets 625 to the open configuration.

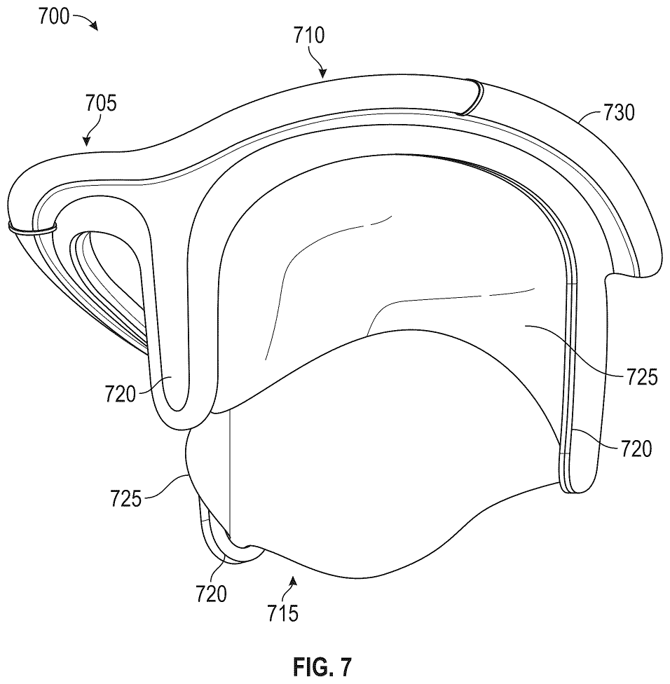

FIG. 7 is a perspective view of a prosthetic heart valve 700, according to another example. The prosthetic heart valve 700 can include a frame 705, a plurality of commissures 720, a plurality of leaflets 725, and a skirt 730. However, other examples of the prosthetic heart valve 700 can include addition or alternative components.

Some examples of the prosthetic heart valve 700 can include the MITRIS RESILIA mitral prosthetic heart valve from Edwards Lifesciences of Irvine, California. Other examples of the prosthetic heart valve can include the INSPIRIS RESILIA aortic prosthetic heart valve, also from Edwards Lifesciences of Irvine, California. However, other examples of the prosthetic heart valve 700 can include different models from different sources.

In the illustrated example, each of the plurality of leaflets 725 can comprise a free edge portion disposed towards an outflow end portion 710 of the prosthetic heart valve 700, wherein the free edge portion is adjacent two lateral portions. Each of the two lateral portions, which extend between an inflow end portion 715 and the outflow end portion 710, are secured to corresponding adjacent ones of the plurality of commissures 720 while the free edge portion remains unattached to the plurality of commissure posts 720 or the frame 705.

In the illustrated example, each of the plurality of leaflets 725 can be biased or shape-set to the open configuration, in which the free edge portion of each of the plurality of leaflets 725 generally follows or tracks the frame 705 in a circumferential direction. In some examples, each of the plurality of leaflets 725 can form a generally bell-shaped curve, wherein the tip of the bell-shaped curve extends outwards towards the circumference of the frame 705 and each end portion of the bell-shaped curve terminates at one of the plurality of commissures 720.

In some examples, in which the prosthetic heart valve 700 is implanted in a native mitral annulus, biasing or shape-setting the plurality of leaflets 725 in the open configuration can beneficially reduce the amount of force needed to open the plurality of leaflets 725, thereby facilitating the actuation of the plurality of leaflets 725 from the closed configuration to the open configuration.

However, in other examples, the prosthetic heart valve 700 can be implanted in an aortic annulus or any other suitable annulus in a patient's heart. If the prosthetic heart valve 700 is implanted in the aortic annulus, the plurality of leaflets 725 can be biased or shape-set to a closed configuration, in which the free edge portions of each of the plurality of leaflets 725 extend into a central flow orifice formed by the frame 705. The plurality of leaflets 725 in the closed configuration can occlude blood flow through the central flow orifice formed by the frame 705. In these examples, biasing or shape-setting the plurality of leaflets 725 in the closed configuration can help counteract the pressure gradient across the aortic annulus that tends to force the plurality of leaflets 725 to the open configuration.

### Examples of the Disclosed Packaging Assembly

The prosthetic heart valve packaging assemblies disclosed throughout this application and the claims can alternatively be referred to as packaging assemblies, packaging units, packages, storage assemblies, or storage units. The disclosed packaging assemblies can be used to hold or store the prosthetic heart valves disclosed elsewhere in the application and in the claims. However, the disclosed packaging assemblies can additionally or alternatively be used to hold or store other prosthetic heart valves. In some examples, the disclosed packaging assemblies can be used in conjunction with other packaging assemblies, surgical equipment, or other apparatuses.

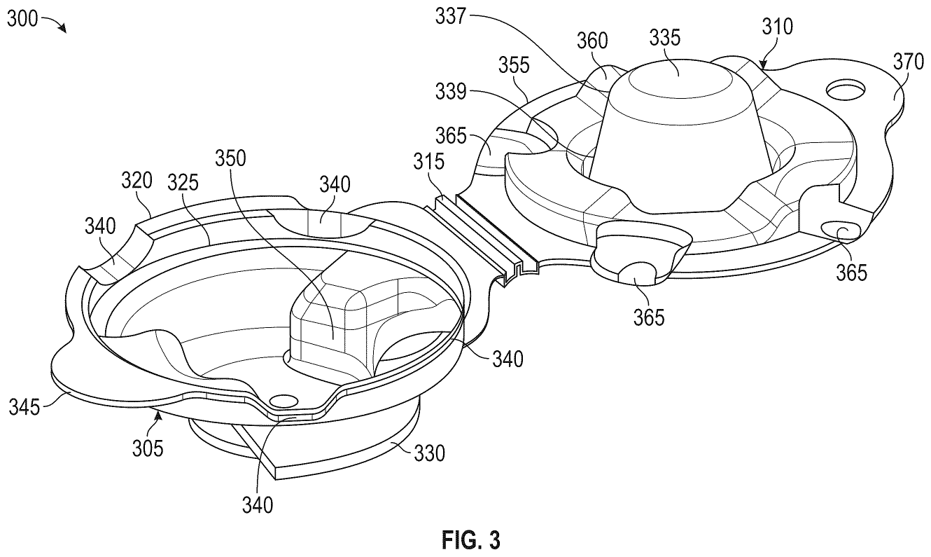

FIG. 3 is a perspective view of a clamshell member 300 for a prosthetic heart valve packaging assembly, according to one example. In some examples, the packaging assembly can comprise the clamshell member 300 and other components such as those disclosed elsewhere in this application and in the claims. In other examples, the packaging assembly can consist solely of the clamshell member 300. However, other examples of the packaging assembly can comprise additional or alternative components.

The clamshell member 300, which can also be referred to as a packaging member, a valve packaging member, or an inner packaging member, can be configured to facilitate dry storage of the prosthetic heart valve in a non-aqueous environment prior to surgical implantation. Additionally or alternatively, the clamshell member 300 can hold the prosthetic heart valve during the sterilization process. Furthermore, the clamshell member 300 can help shape-set the leaflets of the prosthetic heart valve in a desired configuration that can beneficially reduce the amount of force required to open or close a leaflet.

The clamshell member 300 can comprise a lower half 305, an upper half 310, and a hinge 315 connecting the lower half 305 and the upper half 310. However, other examples of the clamshell member 300 can comprise additional or alternative components.

The lower half 305 of the clamshell member 300 can include an annular rim 320 above and surrounding a circular aperture defined by a lower ledge 325. The circular aperture can form a diameter. The diameter can be equal to or larger than a cross-sectional diameter of the prosthetic heart valve, thereby allowing the prosthetic heart valve to pass through the circular aperture.

The lower half 305 can further include a cavity 330 configured to hold, accommodate, and/or store the prosthetic heart valve. The cavity 330 can extend downwards from the lower ledge 325 in a direction opposite the annular rim 320. The cavity 330 can form an enclosed volume with the circular aperture defined by the lower ledge 325 forming an opening into the enclosed volume. The enclosed volume can form a cylindrical, truncated cone, or dome shape. However, other examples of the cavity 330 can form other suitable shapes. In some examples, the cavity 330 can be formed such that the enclosed volume has the same general shape as the prosthetic heart valve, thereby reducing the amount of excess space within the cavity 330. In some examples, the cavity 330 can comprise additional openings, vents, permeable surfaces, or channels to facilitate a movement of fluids or gases during the dehydration or sterilization processes.

In some examples, the lower half 305 can further include one or more lower gas channels 340 configured to permit fluids or gases to permeate the cavity 330 during the sterilization process. In some examples, in which the sterilization process involves exposing the prosthetic heart valve to an ethylene oxide gas, the lower gas channels 340 can be configured to allow the ethylene oxide gas to permeate the clamshell member 300. The lower gas channels 340 can comprise gaps or channels in the annular rim 320, wherein the gaps or channels are configured to permit sterilization gas to cross the annular rim 320 and enter the cavity 330. The lower gas channels 340 can be circumferentially disposed on and radially oriented around the annular rim 320. In some examples, the lower gas channels 340 can be disposed between the annular rim 320 and the lower ledge 325. As illustrated in FIG. 3, the lower half 305 can comprise four lower gas channels 340 spaced approximately 90 degrees apart, wherein the lower gas channels 340 are disposed between the annular rim 320 and the lower ledge 325. However, other examples of the lower half 405 can comprise any suitable number of lower gas channels 340 arranged in any suitable layout.

In some examples, the lower half 305 can further include a lower finger tab 345 configured to facilitate the opening and closing of the clamshell member 300. The illustrated lower finger tab 345 can be a flat, semi-circular tab extending away from the hinge 315. However, other examples of the lower finger tab 345 can have any other suitable shape. The lower finger tab 345 can be disposed on the annular rim 320 opposite the hinge 315. However, the lower finger tab 345 can be located on other suitable locations of the clamshell member 300.

In some examples, the lower half 305 can further include one or more spacers 350 configured to help position the bioprosthetic valve in the cavity 330. The spacers 350 can comprise extrusions extending from a lateral surface and/or the bottom surface of the cavity 330. In some examples, the extrusions can extend from the bottom surface of the cavity 330 to the lower ledge 325. In some examples, the extrusions can form an arc shape that can follow the curvature of the cavity 330.

The upper half 310 of the clamshell member 300 can comprise an upper ledge 355 extending from the hinge 315. The illustrated upper ledge 355 can comprise a flat, substantially circular lid. In other examples, the upper ledge 355 can comprise a lid of any shape that substantially matches the shape and/or outer diameter of the annular rim 320 of the lower half 305.

The upper half 310 can further comprise an inner boss 360 configured to mate with the lower half 305. The inner boss 360 can comprise a cylindrical projection downwardly extending from a bottom surface of the upper ledge 355 and towards the lower half 305. In some examples, the inner boss 360 can comprise a ring-shaped extrusion extending from a central portion of the bottom surface of the upper ledge 355.

In some examples, the outer diameter of the inner boss 360 is approximately equal to an inner diameter of the annular rim 320 and/or the lower ledge 325. This can permit the inner boss 360 to frictionally engage an inner surface of the lower half 305 between the annular rim 320 and the lower ledge 325, thereby frictionally sealing the upper half 310 to the lower half 305.

In some examples, the upper half 310 can further include one or more upper gas channels 365 configured to permit a sterilization gas, e.g., ethylene oxide, to permeate the cavity 330 during the sterilization process. The upper gas channels 365 can comprise channels circumferentially disposed on and radially oriented around the upper ledge 355. The upper gas channels 365 can be equally spaced on the upper ledge 355. As illustrated in FIG. 3, the upper half 310 can comprise four upper gas channels 365 spaced approximately 90 degrees apart. However, other examples of the upper half 310 can comprise any suitable number of upper gas channels 365 arranged in any suitable layout.

In some examples, each of the upper gas channels 365 can align with a respective one of the lower gas channels 340. This can beneficially promote a flow of fluids or gases, e.g., ethylene oxide, into the cavity 330.

In some examples, the inner boss 360 can further comprise cutouts adjacent each upper gas channel 365. The cutouts can be semi-circular cutouts, wherein each semi-circular cutout is aligned with a respective one of the upper gas channels 365, that permit fluid to flow from the upper gas channels 365 and/or the lower gas channels 340 and into the cavity 330.

In some examples, the upper half 310 can further comprise a leaflet biasing element 335. The leaflet biasing element 335, which also can be referred to as a protrusion, an extrusion, a boss, or a paraboloid, can comprise a structure extending from a central portion of the upper ledge 355 towards the lower half 305. The leaflet biasing element 335 can have a substantially paraboloid shape. However, the leaflet biasing element 335 can have any other suitable shape, including a cylinder, a cone, a truncated cone, a dome, or a cuboid.

The leaflet biasing element 335 can have a outer diameter or an outer dimension, wherein the outer diameter or outer dimension can be smaller than the central flow orifice of the prosthetic heart valve. In some examples, the leaflet biasing element 335 can comprise a first portion 337 and a second portion 339. The first portion 337 can be a portion of the leaflet biasing element 335 with a first diameter. The second portion 339 can be a portion of the leaflet biasing element 335 with a second diameter. In some examples, the first diameter is smaller than the second diameter.

The leaflet biasing element 335 can help bias the plurality of leaflets of the prosthetic heart valve in a desired configuration by securing the plurality of leaflets in the desired configuration during a shape-setting process. For example, if the clamshell member 300 is configured to bias the plurality of leaflets in an open configuration, in which the plurality of leaflets can be configured to permit blood flow through the prosthetic heart valve, the leaflet biasing element 335 can be configured to align with the central flow orifice of the prosthetic heart valve.

When the prosthetic heart valve is inserted into the cavity 330 with an outflow end portion of the prosthetic heart valve inserted first, the leaflet biasing element 435 can pass through the inflow end portion of the prosthetic heart valve, enter the central flow orifice, contact the plurality of leaflets, and bias the plurality of leaflets towards a circumferential region of the frame of the prosthetic heart valve when the upper half 310 is closed over the lower half 305. In some examples, the first portion 337 of the leaflet biasing element 335 aligns with the outflow end portion of the prosthetic heart valve, while the second portion 339 of the leaflet biasing element 335 aligns with the inflow end portion. If the plurality of leaflets are biased towards the circumferential region of the frame during the shape-setting process, the plurality of leaflets can become shape-set to the open configuration.

In other examples, the leaflet biasing element 335 can help bias the plurality of leaflets in an intermediate configuration between the open configuration and a closed configuration, in which the plurality of leaflet can be configured to occlude blood flow through the prosthetic heart valve. In these examples, the leaflet biasing element 335 can be smaller in diameter or height than the leaflet biasing element 335 used to bias the plurality of leaflets in the open configuration.

In other examples, the clamshell member 300 can be configured to bias the plurality of leaflets in the closed configuration. In such examples, the clamshell member 300 can comprise a plurality of leaflet biasing elements 335 circumferentially disposed in an intermediate portion of the upper ledge 355. The intermediate portion can be disposed between the center portion and a circumferential edge portion of the upper ledge 355. In some of these examples, each of the plurality of leaflet biasing elements 335 can comprise a structure extending from the intermediate portion of the upper ledge 355 towards the lower half 305. Each of the plurality of leaflet biasing elements 335 can have a substantially paraboloid shape. However, each of the leaflet biasing elements 335 can have a any other suitable shape, including a cylinder, a cone, a truncated cone, a dome, or a cuboid.

In some examples, each of the plurality of leaflet biasing elements 335 can be configured or positioned to be disposed between an outer surface of the leaflet and an inner surface of the frame, thereby securing the leaflet in the closed configuration. In some of these examples, the plurality of leaflet biasing elements 335 can be bent such that the plurality of leaflet biasing elements 335 can be bent radially inwards or outwards to configure or position the plurality of leaflet biasing elements 335 against the outer surface of the leaflets. In some of these examples, the plurality of leaflet biasing elements 335 can be formed from a material that reduces a risk of rupturing the plurality of leaflets. In some of these examples, the plurality of leaflet biasing elements 335 are formed from the same material as the rest of the clamshell member 300.

In some examples, the upper half 310 can further include an upper finger tab 370 configured to facilitate the opening and closing of the clamshell member 300. The illustrated upper finger tab 370 can be a flat, semi-circular tab extending away from the hinge 315. In some of these examples, the upper finger tab 370 can be the same size and shape as the lower finger tab 345. However, other examples of the upper finger tab 370 can have any other suitable shape.

The upper finger tab 370 can be disposed on the upper ledge 355 opposite the hinge 315. In some of these examples, the upper finger tab 370 can be disposed on the upper half 310 such that the upper finger tab 370 is aligned with the lower finger tab 345. However, the upper finger tab 370 can be located on other suitable locations of the upper half.

The hinge 315 can connect the lower half 305 to the upper half 310. In some examples, the hinge 315 can connect to the lower ledge 325 and the upper ledge 355. In some examples, the hinge 315 can be a living hinge formed from the same material as the lower half 305 and the upper half 310. However, other hinges 315 can have different designs and can be formed of different materials.

In some examples, the clamshell member 300 can be constructed of a molded material, such as a polyethylene terephthalate glycol (PETG). However, other examples of the clamshell member 300 can be constructed of another thermoplastic, plastic, metal, or any other suitable material. Furthermore, some examples of the clamshell member 300 can be 3D-printed and can thus be constructed of a thermoplastic, fiber, metal, or any other material for 3D-printing.

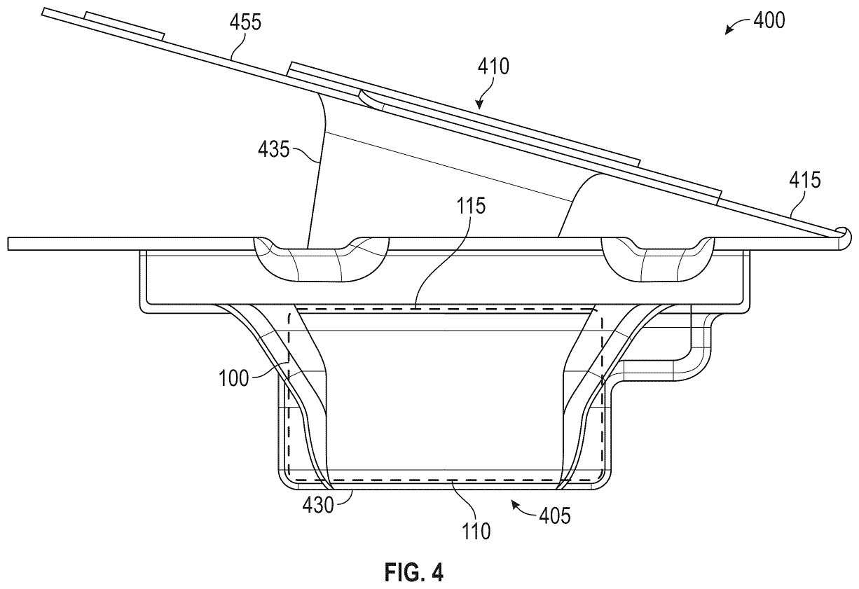

FIG. 4 is a side view of a clamshell member 400, according to one example. The clamshell member 400 comprises a lower half 405, and upper half 410, and a hinge 415 connecting the lower half 405 and the upper half 410. However, other examples of the clamshell member 400 can comprise additional or alternative components.

The lower half 405, which can alternatively be referred to as a clamshell tray, a valve tray, a lower tray, or an internal tray, can comprise a cavity 430 extending opposite the upper half 410. The cavity 430 can be configured to accept the prosthetic heart valve 100. The illustrated clamshell member 400 can be configured to accept the prosthetic heart valve 100 of FIG. 1. In the illustrated example, the prosthetic heart valve 100 fills a portion of the cavity 430 defined by the dashed line. However, the clamshell member 400 can be configured to accept other example prosthetic heart valves with other shapes and sizes.

The upper half 410 can comprise an upper ledge 455 and a leaflet biasing element 435. The upper ledge 455 can form a lid configured to cover an opening or aperture of the lower half 405. The upper half 410 can further comprise a leaflet biasing element 435 extending from a central portion of the upper ledge 455. In some examples, disposing the leaflet biasing element 435 on the upper half 410 can promote manufacturability of the clamshell member 400.

The leaflet biasing element 435, which also can be referred to as a protrusion, an extrusion, a boss, or a paraboloid, can be configured to bias one or more of the plurality of leaflets of the prosthetic heart valve 100 in a desired configuration. If the clamshell member 400 is configured to bias the plurality of leaflets in the open configuration, the clamshell member 400 can comprise the leaflet biasing element 435 extending from a center portion of a bottom surface of the upper ledge 455. In some examples, the center portion of the bottom surface of the upper ledge 455 can align with a central flow orifice of the prosthetic heart valve 100.

The leaflet biasing element 435 can comprise a structure extending from the central portion of the bottom surface of the upper ledge 455 towards the lower half 405. The leaflet biasing clement 435 can have a substantially paraboloid shape. However, the leaflet biasing element 435 can have any other suitable shape, including a cylinder, a cone, a truncated cone, a dome, or a cuboid. The leaflet biasing element 435 can have a outer diameter or an outer dimension, wherein the outer diameter or outer dimension can be smaller than the central flow orifice of the prosthetic heart valve 100.

The leaflet biasing element 435 can help bias the plurality of leaflets in a desired configuration by securing the leaflets in the desired configuration during a shape-setting process. For example, if the clamshell member 400 is configured to bias the plurality of leaflets in the open configuration, the leaflet biasing element 435 can be configured to align with the central flow orifice of the prosthetic heart valve 100. When the prosthetic heart valve is inserted into the cavity 430 with the outflow end portion 110 of the prosthetic heart valve 100 disposed towards a bottom surface of the cavity 430 and the inflow end portion 115 of the prosthetic heart valve 100 disposed towards the upper half 410, the leaflet biasing element 435 can pass through the inflow end portion of the prosthetic heart valve 100, enter the central flow orifice, contact the plurality of leaflets, and bias the plurality of leaflets towards a circumferential region of a frame of the prosthetic heart valve 100. If the plurality of leaflets are biased towards the circumferential region of the frame during the shape-setting process, the plurality of leaflets can become shape-set to the open configuration.

In other examples, the leaflet biasing element 435 can help bias the plurality of leaflets in the intermediate configuration between the open configuration and the closed configuration. In these examples, the leaflet biasing element 435 can be shorter or narrower than the leaflet biasing element 435 used to bias the plurality of leaflets in the open configuration.

In other examples, the clamshell member 400 can be configured to bias the plurality of leaflets in the closed configuration. In such examples, the clamshell member 400 can comprise a plurality of leaflet biasing elements 435 circumferentially disposed in an intermediate portion of the bottom surface of the upper ledge 455. The intermediate portion can be disposed between the center portion and a circumferential edge portion of the bottom surface of the upper ledge 455.

In some of these examples, each of the plurality of leaflet biasing elements 435 can comprise a structure extending from the intermediate portion of the bottom surface of the upper ledge 455 towards the lower half 405. Each of the plurality of leaflet biasing elements 435 can have a substantially paraboloid shape. However, each of the leaflet biasing elements 435 can have any other suitable shape, including a cylinder, a cone, a truncated cone, a dome, or a cuboid.

In some examples, each of the leaflet biasing elements 435 can be configured or positioned to be disposed between an outer surface of the leaflet and an inner surface of the frame, thereby securing the leaflet in the closed configuration. In some of these examples, the leaflet biasing elements 435 can be bent such that the leaflet biasing elements 435 contact the outer surface of the leaflets. In some of these examples, the plurality of leaflet biasing elements 435 can be formed from a material that reduces a risk of rupturing the plurality of leaflets. In some of these examples, the leaflet biasing elements 435 and be formed from the same material as the rest of the clamshell member 400.

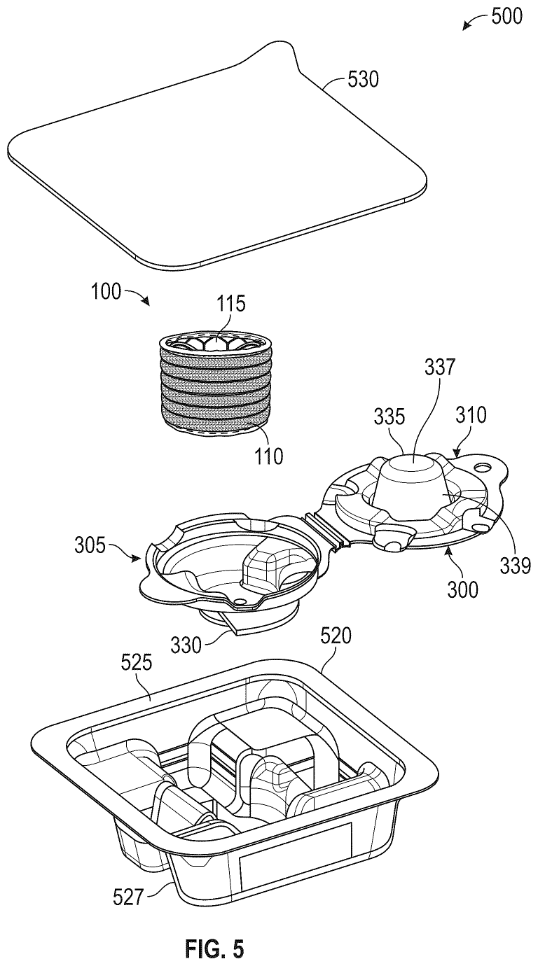

FIG. 5 illustrates an exploded view of a prosthetic heart valve packaging assembly 500, according to one example. The packaging assembly 500 can facilitate the sterile and dry storage of a prosthetic heart valve 100 prior to surgical implantation. The packaging assembly 500 can comprise a clamshell member 300, a storage tray 520, and a lid 530. The prosthetic heart valve 100 is not necessarily part of the packaging assembly 500 but is included in FIG. 5 for context. However, other examples of the packaging assembly 500 can comprise additional or alternative components.

The prosthetic heart valve 100 illustrated in FIG. 5 can be the same valve depicted in FIG. 1. However, in other examples, the prosthetic heart valve packaging assembly 500 can be configured to store the prosthetic heart valve 600 depicted in FIG. 6, the prosthetic heart valve 700 of FIG. 7, any prosthetic heart valve depicted in the application or in the claims, or any other suitable prosthetic heart valve.

The clamshell member 300 can facilitate the shape-setting of a plurality of leaflets of the prosthetic heart valve 100. The clamshell member 300 can comprise a lower half 305 with a cavity 330 sized to accept the prosthetic heart valve 100. The clamshell 300 can further comprise an upper half 310 with a leaflet biasing element 335 to bias the plurality of leaflets in a desired configuration. Biasing the plurality of leaflets during a shape-setting process can beneficially shape-set the leaflets in the desired configuration, thereby improving the ability of the plurality of leaflets to actuate between the open configuration and the closed configuration during normal valve operation. Furthermore, some examples of the clamshell member 300 can hold the prosthetic heart valve 100 during the sterilization process. In some examples, the clamshell member 300 can be substantially similar to the clamshell member 400 depicted in FIG. 4 or other clamshell members disclosed in the application and the claims.

In the illustrated example, the prosthetic heart valve 100 can be inserted into the clamshell member 300 such that the outflow end portion 110 of the prosthetic heart valve 100 faces down or towards a bottom surface of the cavity 330 and the inflow end portion 115 of the prosthetic heart valve 100 faces up or towards the upper half 310. This orientation can allow the leaflet biasing element 335, which is disposed on the upper half 310 of the clamshell member 300, to engage the plurality of leaflets.

In other examples, in which the leaflet biasing element 335 is disposed on a bottom surface or a lower half of the clamshell member 300, the prosthetic heart valve 100 can be inserted such that the inflow end portion 115 of the prosthetic heart valve 100 can face down or towards a bottom surface of the clamshell member 300.

The storage tray 520, which can also be referred to as an outer packaging member, can be configured to hold the clamshell member 300 after the prosthetic heart valve 100 has been dehydrated and/or shape-set to the clamshell member 300. The storage tray 520 can comprise a flat, horizontal outer rim 525 that can surround a cavity 527. The cavity 527 can extend downwardly from the outer rim 525 and can be sized to accept the clamshell member 300. In some examples, the cavity 527 can be configured to accept the entirety of the clamshell member 300.

The storage tray 520 can be a molded material, such as a polyethylene terephthalate copolymer (PETG), that provides rigidity and protection from jostling and external pressures. However, the storage tray 520 can be made of any suitable material, including plastics, polymers, or 3D-printing materials.

In some examples, the storage tray 520 can resemble the storage trays disclosed in U.S. Patent Publication No. 2011/0214398, filed on Mar. 2, 2011, and U.S. Patent Publication No. 2012/0158128, filed on Dec. 13, 2011.

The lid 530 can be configured to seal the storage tray 520. More specifically, the outer rim 525 of the storage tray 520 can form a flange to which a band of adhesive (not pictured) on the lid 530 may be adhered., The lid 530 can closely dimensioned to the perimeter of the outer rim 525, and the band of adhesive can be a pressure-seal or a heat seal adhesive to facilitate sealing under pressure and/or temperature. The lid 530 can be formed from a breathable or gas-permeable material to provide for gas sterilization of the contents sealed within the storage tray 520, e.g., the prosthetic heart valve 100. In some examples, various Tyvek materials from DuPont may be used to form the lid 530. In other examples, the lid 530 can be formed of a material that permits sterilization of the prosthetic heart valve using ethylene oxide, which gradually passes through the lid 530 to the cavity 527 of the storage tray 520 but prevents an ingress of microorganisms.

In some examples, the lid 530 can resemble the lids disclosed in U.S. Patent Publication No. 2011/0214398, filed on Mar. 2, 2011, and U.S. Patent Publication No. 2012/0158128, filed on Dec. 13, 2011.

In other examples, the lid 530 can be gas-impermeable to prevent gases from entering the storage tray 520 and contaminating the sterilized prosthetic heart valve.

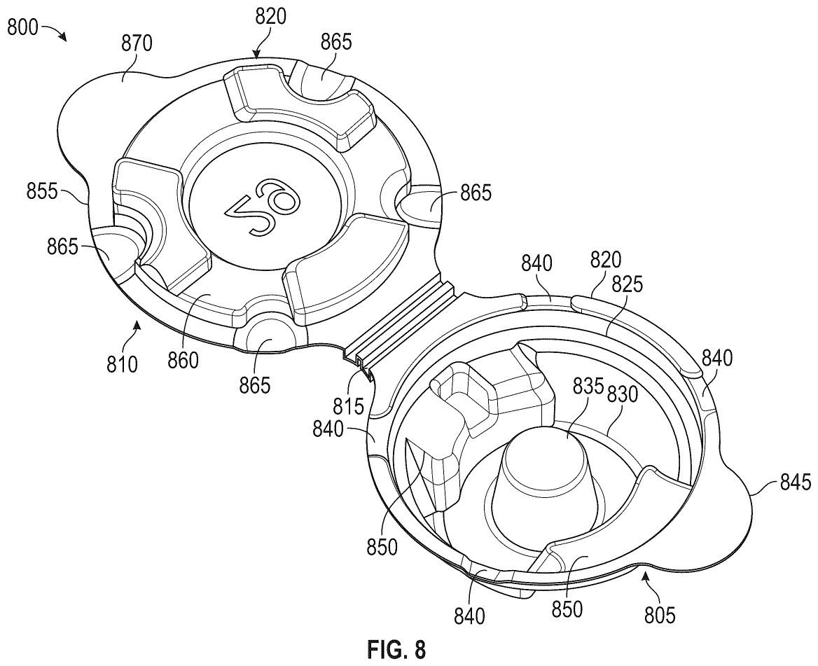

FIG. 8 is a perspective view of a clamshell member 800 for a prosthetic heart valve packaging assembly, according to one example. In some examples, the packaging assembly can comprise the clamshell member 800 and other components such as those disclosed elsewhere in this application and in the claims. In other examples, the packaging assembly can consist solely of the clamshell member 300. However, other examples of the packaging assembly can comprise additional or alternative components.

The clamshell member 800 can be configured to facilitate dry storage of the prosthetic heart valve in a non-aqueous environment prior to surgical implantation. Additionally or alternatively, the clamshell member 800 can hold the prosthetic heart valve during the sterilization process. Furthermore, the clamshell member 800 can help shape-set the leaflets of the prosthetic heart valve in a desired configuration that can beneficially reduce the amount of force required to open or close the leaflets.

The clamshell member 800 can comprise a lower half 805, an upper half 810, and a hinge 815 connecting the lower half 805 and the upper half 810. However, other examples of the clamshell member 800 can comprise additional or alternative components.

The clamshell member 800 depicted in FIG. 8 can have a similar design as the clamshell member 300 depicted in FIG. 3. However, the clamshell member 800 of FIG. 8 can comprise a leaflet biasing element 835 extending from a central portion of a bottom internal surface of the cavity 830 towards the annular rim 820. The leaflet biasing element 835, which also can be referred to as a protrusion, an extrusion, a boss, or a paraboloid, can have a substantially paraboloid shape. However, the leaflet biasing element 835 can have any other suitable shape, including a cylinder, a cone, a truncated cone, a dome, or a cuboid. The leaflet biasing element 835 can have a outer diameter or an outer dimension, wherein the outer diameter or outer dimension can be smaller than the central flow orifice of the prosthetic heart valve.

The leaflet biasing element 835 can help bias the plurality of leaflets of the prosthetic heart valve in a desired configuration by securing the plurality of leaflets in the desired configuration during a shape-setting process. For example, if the clamshell member 800 is configured to bias the plurality of leaflets in an open configuration, in which the plurality of leaflets can be configured to permit blood flow through the prosthetic heart valve, the leaflet biasing element 835 can be configured to align with the central flow orifice of the prosthetic heart valve. When the prosthetic heart valve is inserted into the cavity 830 with an inflow end portion of the prosthetic heart valve inserted first, the leaflet biasing element 835 can pass through the inflow end portion of the prosthetic heart valve, enter the central flow orifice, contact the plurality of leaflets, and bias the plurality of leaflets towards a circumferential region of the frame of the prosthetic heart valve. If the plurality of leaflets are biased towards the circumferential region of the frame during the shape-setting process, the plurality of leaflets can become shape-set to the open configuration.

In other examples, the leaflet biasing element 835 can help bias the plurality of leaflets in an intermediate configuration between the open configuration and a closed configuration, in which the plurality of leaflets can be configured to occlude blood flow through the prosthetic heart valve. In these examples, the leaflet biasing element 835 can be smaller in diameter or height than the leaflet biasing element 835 used to bias the plurality of leaflets in the open configuration.

In other examples, the clamshell member 800 can be configured to bias the plurality of leaflets in the closed configuration. In such examples, the clamshell member 800 can comprise a plurality of leaflet biasing elements 835 circumferentially disposed in an intermediate portion of the bottom internal surface of the cavity 830. The intermediate portion can be disposed between the center portion and a circumferential edge portion of the bottom internal surface of the cavity 830. In some of these examples, each of the plurality of leaflet biasing elements 835 can comprise a structure extending from the intermediate portion of the upper ledge 855 towards the lower half 805. Each of the plurality of leaflet biasing elements 835 can have a substantially paraboloid shape. However, each of the leaflet biasing elements 835 can have any other suitable shape, including a cylinder, a cone, a truncated cone, a dome, or a cuboid.

In some examples, each of the plurality of leaflet biasing elements 835 can be configured or positioned to be disposed between an outer surface of the leaflet and an inner surface of the frame, thereby securing the leaflet in the closed configuration. In some of these examples, the plurality of leaflet biasing elements 835 can be bent such that the plurality of leaflet biasing elements 835 contact the outer surface of the leaflets. In some of these examples, the plurality of leaflet biasing elements 835 can be formed from a material that reduces a risk of rupturing the plurality of leaflets. In some of these examples, the plurality of leaflet biasing elements 835 can be formed from the same material as the rest of the clamshell member 800.

For convenience, similar reference numbers can describe similar components. For example, lower ledge 825 can be similar to the lower ledge 325, lower gas channels 840 can be similar to lower gas channels 340, lower finger tab 845 can be similar to the lower finger tab 345, spacers 850 can be similar to spacers 350, inner boss 860 can be similar to the inner boss 360, upper gas channels 865 can be similar to upper gas channels 865, and upper finger tab 870 can be similar to the upper finger tab 370.

In some examples, the clamshell member 800 can be constructed of a molded material, such as a polyethylene terephthalate glycol (PETG). However, other examples of the clamshell member 800 can be constructed of another thermoplastic, plastic, metal, or any other suitable material. Furthermore, some examples of the clamshell member 800 can be 3D-printed and can thus be constructed of a thermoplastic, fiber, metal, or any other material for 3D-printing.

### Examples of the Disclosed Methodology

The methods and processes described in this application and in the claims can be used to shape-set a plurality of leaflets of a prosthetic heart valve in a desired configuration. The prosthetic heart valves disclosed in relation to this method can be those described elsewhere in this application and in the claims. However, the disclosed methods can involve other suitable prosthetic heart valves. The prosthetic heart valves can be configured for implantation in a mitral annulus, a pulmonary annulus, an aortic annulus, and/or a tricuspid annulus. However, this method can be applied to prosthetic heart valves configured for surgical implantation at other sites.

The desired configuration of the plurality of leaflets can be an open configuration that permits blood flow through the prosthetic heart valves, a closed configuration that occludes blood flow through the prosthetic heart valves, or an intermediate configuration between the open configuration and the closed configuration.

In some examples, the disclosed methods can be performed as part of an assembly or fabrication process for the prosthetic heart valves. In other examples, the disclosed methods can be performed on previously assembled prosthetic heart valves whose bioprosthetic tissue has not yet been subjected to the disclosed methods.

Although the disclosed methods can be described in a particular or sequential order for convenient presentation, it should be understood that this manner of description encompasses rearrangement, unless a particular ordering is required by specific language set forth below. For example, methods described sequentially may in some cases be rearranged or performed concurrently. The actual methods that correspond to these terms may vary depending on the particular implementation and are readily discernible by one of ordinary skill in the art.

The shape-setting process can first comprise a step of dehydrating the prosthetic heart valve. Dehydration beneficially allows the prosthetic heart valves to be stored in a non-fluid environment, which eliminates the need for the prosthetic heart valves to be stored in formaldehyde, glutaraldehyde, or other aqueous storage solutions. The non-fluid environment can be an environment in which the presence of water or other substances is limited to approximately the content of such substances in ambient air (as more precisely defined by the relative humidity). Furthermore, dry storage can be beneficial because the prosthetic heart valves do not need to be rinsed prior to surgical implantation.

One method of bioprosthetic tissue dehydration includes treating the plurality of leaflets with a non-aqueous treatment solution comprising a polyhydric alcohol and a C₁-C₃ alcohol. In some of these examples, the polyhydric alcohol can be a glycerol and the C₁-C₃ alcohol can be selected from the group consisting of ethanol, n-propanol, 2-propanol or a mixture thereof. The polyhydric alcohol concentration can be 40-95% and the C₁-C₃ alcohol concentration can be 5-60% by volume. The prosthetic heart valve can be immersed in the non-aqueous treatment solution to displace water within the bioprosthetic tissue with glycerol.

Once the bioprosthetic tissue has been sufficiently exposed to the non-aqueous treatment solution, the bioprosthetic tissue can be removed from the non-aqueous treatment solution and exposed to ambient air or an inert environment, e.g., nitrogen, at standard room temperature and humidity so as not to adversely affect tissue properties. the drying can be performed in a clean room or in a laminar flow bench at ambient room conditions for about 1-4 hours.

Other examples of bioprosthetic tissue dehydration processes can be found in U.S. Patent Application Publication No. 2008/0102439, filed on Oct. 23, 2007. Further exemplary bioprosthetic tissue dehydration processes can include the RESILIA® bioprosthetic tissue treatment process from Edwards Lifesciences in Irvine, California. However, other suitable bioprosthetic tissue treatment processes can be used.

In some of these examples, the plurality of leaflets can be pretreated with an calcification mitigant prior to the dehydration process to reduce in vivo calcification of the bioprosthetic tissue. The calcification mitigant can reduce calcification by blocking, removing, or altering functional sites that attract calcium. In some examples, the calcification mitigant can be an aldehyde capping agent such as ethanolamine. However, in other examples, other calcification mitigants can be used. Further exemplary anti-calcification processes can be found in U.S. Patent Application Publication No. 2009/0164005, filed on Dec. 18, 2008.

After the prosthetic heart valve has been dehydrated, the plurality of leaflets of the prosthetic heart valve are biased to the desired configuration.

After the prosthetic heart valve has been dehydrated, the leaflets of the prosthetic heart valve can be secured in the desired configuration prior to shape-setting. In some examples, the plurality of leaflets can be secured by inserting the prosthetic heart valve into one of the packaging assemblies disclosed in this application and in the claims. In examples in which the leaflet biasing element is disposed on in a cavity of a lower half of the packaging assembly, the plurality of leaflets are secured in the desired configuration when the prosthetic heart valve is inserted into the cavity of the lower half. In examples in in which the leaflet biasing element is disposed on a lid of an upper half of the packaging assembly, the plurality of leaflets are secured in the desired configuration when the lid is closed over the lower half. However, other suitable packaging assemblies can be used to secure the plurality of leaflets.

In other examples, the plurality of leaflets can be secured in the desired configuration using a jig, a clamp, an insert, or any other suitable support structure configured to contact the plurality of leaflets and restrain the plurality of leaflets in the desired configuration. In other further examples, the plurality of leaflets can be secured by temporarily securing the plurality of leaflets in the desired configuration using tape, adhesive, or sutures. However, any method suitable for securing the plurality of leaflets in the desired configuration during the shape-setting process can be used.

In some examples, in which the prosthetic heart valve can be implanted in a mitral annulus of a heart surgery patient, the plurality of leaflets can be biased to the open configuration in which a free edge of each of the plurality of leaflets generally follows a circumference of a frame of the prosthetic heart valve. In other examples, in which the prosthetic heart valve can be implanted in an aortic annulus of a heart surgery patient, the plurality of leaflets can be biased to the closed configuration in which a free edge of each of the plurality of leaflets extends towards a central flow orifice of the prosthetic heart valve. In further examples, the plurality of leaflets can be biased to an intermediate configuration between the open configuration and the closed configuration.

After the plurality of leaflets have been biased to the desired configuration, the plurality of leaflets can be shape-set to the desired configuration. In some examples, shape-setting can occur as a result of a particular combination of dehydration and sterilization processes.

However, shape-setting can occur after different combinations of dehydration, sterilization, or other processes.

In some examples, the plurality of leaflets can be shape-set after the dehydration process and during the sterilization process. In some of these examples, the plurality of leaflets can become shape-set when the dry bioprosthetic valve is exposed to ethylene oxide gas and after it is dehydrated using the RESILIA® bioprosthetic tissue treatment process from Edwards Lifesciences of Irvine, California.

In some examples, a sterilization process using ethylene oxide gas can comprise exposing the prosthetic heart valve to an environment comprising 10% ethylene oxide and 90% hydrochlorofluorocarbon at a chamber pressure of 8 to 10 psig at a temperature of 38° C. for 24 hours or at a temperature of 54-57° C. for 130 minutes. However, other compositions of gases can be used to sterilize the prosthetic heart valve. Furthermore, the sterilization process can occur under different temperatures and pressures. In other examples, the prosthetic heart valve can be sterilized by exposure to ionizing radiation, including gamma and electron beam radiation. Finally, it should be understood that the shape-setting process can conceivably occur during different sterilization processes.

### ADDITIONAL EXAMPLES

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A packaging assembly for storing a prosthetic heart valve, the packaging assembly comprising:
a valve tray comprising an annular rim and a cavity extending away from the annular rim, wherein the cavity is configured to accept the prosthetic heart valve;
a lid configured to cover the annular rim; and
a leaflet biasing element disposed on a central portion of the lid, wherein:
   the leaflet biasing element comprises an extrusion with a paraboloid shape,
   the leaflet biasing element extends from a lower surface of the lid towards the valve tray, and
   the leaflet biasing element is configured to extend at least partially into an inflow end portion of the prosthetic heart valve and secure a plurality of leaflets of the prosthetic heart valve in a desired configuration when the prosthetic heart valve is inserted in the cavity and the lid is closed over the annular rim.

Example 2. A packaging assembly for storing a prosthetic heart valve, the packaging assembly comprising:
a valve tray configured to accept the prosthetic heart valve; and
a leaflet biasing element configured to protrude into the valve tray, wherein the leaflet biasing element is configured to secure a plurality of leaflets of the prosthetic heart valve in a desired configuration.

Example 3. The packaging assembly of any of the above examples, wherein the leaflet biasing element is configured to secure the plurality of leaflets of the prosthetic heart valve in an open configuration.

Example 4. The packaging assembly of any of the above examples, wherein the leaflet biasing element is configured to extend at least partially through a central flow orifice of the prosthetic heart valve to contact the plurality of leaflets.

Example 5. A packaging assembly for storing a prosthetic heart valve, the clamshell packaging assembly comprising:
a valve tray comprising:
   an annular rim,
   a cavity extending in a direction away from the annular rim, wherein the cavity is configured to accept the prosthetic heart valve, and
   a leaflet biasing element disposed on a central portion of a bottom surface of the cavity, wherein the leaflet biasing element extends from the bottom surface towards the annular rim, and wherein the leaflet biasing element is configured to secure a plurality of leaflets of the prosthetic heart valve in a desired configuration when the prosthetic heart valve is seated in the cavity; and
a lid configured to cover the annular rim.

Example 6. The packaging assembly of any of the above examples, wherein the tray further comprises a plurality of lower gas channels circumferentially disposed on the annular rim and projecting in a radial direction, wherein the plurality of lower gas channels are configured to permit gas flow into the cavity.

Example 7. The packaging assembly of any of the above examples, wherein the lid further comprises a plurality of upper gas channels circumferentially disposed on the lid and projecting in a radial direction.

Example 8. The packaging assembly of any of the above examples, wherein each of the upper gas channels is configured to align with a respective one of the lower gas channels when the lid is closed over the annular rim.

Example 9. A packaging assembly for storing a prosthetic heart valve, the packaging assembly comprising:
a first portion comprising an annular rim and a cavity extending in a direction away from the annular rim, wherein the cavity is configured to accept the prosthetic heart valve; and
a second portion comprising a lid configured to cover the annular rim and a leaflet biasing element extending from a central portion of the lid towards the lower half, wherein the leaflet biasing element is configured to secure a plurality of leaflets of the prosthetic heart valve in a desired configuration when the prosthetic heart valve is inserted into the cavity and the second portion is closed over the first portion.

Example 10. The packaging assembly of any of the above examples, wherein the plurality of leaflets of the prosthetic heart valve are biased to an open configuration.

Example 11. The packaging assembly of any of the above examples, wherein the leaflet biasing element is configured to extend at least partially through a central flow orifice of the prosthetic heart valve when the prosthetic heart valve is inserted in the cavity.

Example 12. The packaging assembly of any of the above examples, wherein the leaflet biasing element comprises an extrusion with a paraboloid shape.

Example 13. The packaging assembly of any of the above examples, wherein the packaging assembly further comprises a hinge connecting the first portion and the second portion.

Example 14. A packaging assembly for storing a prosthetic heart valve, the packaging assembly comprising:
an inner packaging member comprising:
   a first portion comprising a circular aperture and a cavity extending downwardly from the circular aperture, wherein the cavity is configured to accept the prosthetic heart valve;
   a second portion comprising a lid configured to cover the circular aperture; and
   a paraboloid-shaped leaflet biasing element extending into the cavity, wherein the leaflet biasing element is configured to secure a plurality of leaflets into a desired configuration; and
   a living hinge coupling the first portion to the second portion;
an outer packaging member configured to accept the inner packaging member; and
a gas-permeable lid configured to seal the outer packaging member when the inner packaging member is inserted into the outer packaging member.

Example 15. The packaging assembly of any of the above examples, wherein the first portion further comprises a lower finger tab extending from the circular aperture and disposed opposite the hinge.

Example 16. The packaging assembly of any of the above examples, wherein the second portion further comprises an upper finger tab extending from the lid and disposed opposite the hinge.

Example 17. The packaging assembly of any of the above examples, wherein the upper finger tab is aligned with the lower finger tab.

Example 18. The packaging assembly of any of the above examples, wherein the first portion further comprises a plurality of spacers circumferentially disposed on an inner lateral surface of the cavity.

Example 19. A packaging assembly for storing a prosthetic heart valve, the packaging assembly comprising:
an inner packaging member comprising a first portion and a second portion, wherein:
the first portion comprises an annular rim surrounded by a circular aperture; a lower ledge defined by and disposed beneath the circular aperture; a first cavity extending downwardly from the lower ledge and configured to accept the prosthetic heart valve; a plurality of radially-extending lower gas channels disposed on the annular rim; and a lower finger tab radially extending from the annular rim, and
the second portion comprises a lid configured to cover the circular aperture; a leaflet biasing element extending from a central portion of the lid towards the first portion, wherein the leaflet biasing element extends at least partially into the first cavity to secure a plurality of leaflets of the prosthetic heart valve in a desired configuration; a plurality of radially-extending upper gas channels disposed on the lid, wherein each of the upper gas channels is configured to align with a respective one of the lower gas channels; and an upper finger tab extending from the lid and aligned with the lower finger tab;
a storage tray comprising an open end and a second cavity extending from the open end, wherein the second cavity is configured to accept the clamshell; and
a gas-permeable lid configured to fit over the open end of the storage tray, wherein the gas-permeable lid is configured to permit gas to enter the valve second cavity but prevent ingress of microorganisms.

Example 20. The packaging assembly of any of the above examples, wherein the second portion further comprises a circular boss extruded from the lid.

Example 21. The packaging assembly of any of the above examples, wherein the circular boss is configured to frictionally engage an inner surface of the first cavity between the annular rim and the lower ledge.

Example 22. The packaging assembly of any of the above examples, wherein the inner packaging member is formed of a gas-permeable material.

Example 23. The packaging assembly of any of the above examples, wherein the inner packaging member is formed of polyethylene terephthalate glycol (PETG).

Example 24. The packaging assembly of any of the above examples, wherein the inner packaging member is formed of polypropylene (PP).

Example 25. The packaging assembly of any of the above examples, wherein the gas-permeable lid is formed from TYVEK.

Example 26. The packaging assembly of any of the above examples, wherein an inflow end of the prosthetic heart valve faces the second portion when the prosthetic heart valve is seated in the clamshell cavity.

Example 27. A method of biasing a plurality of leaflets of a prosthetic heart valve in a desired configuration, the method comprising:
dehydrating the prosthetic heart valve;
securing the plurality of leaflets in the desired configuration, wherein the desired configuration is configured to reduce retrograde flow through the prosthetic heart valve; and
sterilizing the prosthetic heart valve.

Example 28. The method of any of the above examples, wherein the prosthetic heart valve is dehydrated by an exposure to a glycerol/ethanol mixture.

Example 29. The method of any of the above examples, wherein the plurality of leaflets are secured in an open configuration.

Example 30. The method of any of the above examples, wherein the plurality of leaflets are secured in a closed configuration.

Example 31. The method of any of the above examples, wherein the prosthetic heart valve is sterilized by an exposure to an ethylene oxide gas.

Example 32. A method of shape-setting a plurality of leaflets of a prosthetic heart valve to a desired configuration, comprising:
dehydrating the plurality of leaflets;
inserting the prosthetic heart valve into a packaging member, wherein the package member comprises:
   a cavity configured to accept the prosthetic heart valve;
   a lid configured to seal the cavity; and
   a leaflet biasing element disposed on a central portion of a bottom surface of the lid, wherein the leaflet biasing element is configured to extend into the cavity, and wherein the leaflet biasing element is configured to secure the plurality of leaflets in the desired configuration;
closing the lid such that the leaflet biasing element engages the plurality of leaflets; and sterilizing the prosthetic heart valve.

Example 33. The method of claim 32, wherein the prosthetic heart valve is dehydrated by exposing the prosthetic heart valve to a glycerol/ethanol mixture.

Example 34. The method of claim 32, wherein the prosthetic heart valve is sterilized by exposing the prosthetic heart valve to an ethylene oxide gas.

Example 35. The method of any of the above examples, wherein the leaflet biasing element is configured to engage the plurality of leaflets in an open configuration.

Example 36. The method of any of the above examples, wherein the leaflet biasing element is configured to align with a central flow orifice of the prosthetic heart valve.

Example 37. The method of any of the above examples, wherein the leaflet biasing element is configured to partially extend into the central flow orifice when the lid is closed.

Example 38. A method of shape-setting a prosthetic heart valve comprising:
dehydrating a plurality of leaflets of the prosthetic heart valve by treating the prosthetic heart valve with a non-aqueous treatment solution comprising a glycerol and an alcohol;
removing a portion of the non-aqueous treatment solution from the plurality of leaflets;
inserting a leaflet biasing element at least partially into a central flow orifice formed by the prosthetic heart valve, wherein the leaflet biasing element is configured to secure the plurality of leaflets towards a circumferential portion of the central flow orifice; and
sterilizing the prosthetic heart valve by exposing the prosthetic heart valve to an ethylene oxide gas.

Example 39. A method of fabricating a prosthetic heart valve, wherein the prosthetic heart valve comprises a plurality of leaflets formed from bovine pericardium, the method comprising:
immersing at least the plurality of leaflets in a non-aqueous treatment solution comprising a glycerol and a C₁-C₃ alcohol selected from the group consisting of methanol, ethanol, n-propanol, 2-propanol and mixtures thereof, wherein the glycerol is 60-95% by volume;
removing a portion of the non-aqueous treatment solution from the plurality of leaflets;
inserting the prosthetic heart valve into a gas-permeable packaging member, wherein the gas-permeable packaging member is configured to secure the plurality of leaflets in a desired configuration; and
sterilizing the prosthetic heart valve by exposing the gas-permeable packaging member to an ethylene oxide gas.

Example 40. A prosthetic heart valve comprising:
an annular frame extending from an inflow end portion towards an outflow end portion of the prosthetic heart valve;
a plurality of commissure supports circumferentially disposed around the annular frame and projecting towards the outflow end portion; and
a plurality of leaflets secured to the plurality of commissure supports, wherein:
   the plurality of leaflets are formed from bovine pericardium tissue,
   each of the plurality of leaflets comprises a free edge portion between two lateral portions,
   each of the plurality of leaflets is biased to an open configuration in which the free edge portion generally follows the annular frame in a circumferential direction,
   the two lateral portions are secured to adjacent ones of the plurality of commissure supports, and
   the prosthetic heart valve is configured to be stored in a dry state.

Example 41. A prosthetic heart valve comprising:
an annular frame extending from an inflow end portion towards an outflow end portion of the prosthetic heart valve;
a plurality of commissure supports circumferentially disposed around the annular frame and projecting towards the outflow end portion; and
a plurality of leaflets secured to the plurality of commissure supports, wherein the plurality of leaflets are biased to a desired configuration,
wherein the prosthetic heart valve is configured to be stored in a dry state.

Example 42. The prosthetic heart valve of any of the above examples, wherein the plurality of leaflets are biased to an open configuration.

Example 43. The prosthetic heart valve of any of the above examples, wherein the plurality of leaflets are biased to a closed configuration.

Example 44. The prosthetic heart valve of any of the above examples, wherein the plurality of leaflets are formed from bioprosthetic tissue.

Example 45. The prosthetic heart valve of any of the above examples, wherein the plurality of leaflets are formed from bovine pericardium tissue.

Example 46. A prosthetic heart valve comprising:
an annular frame extending from an inflow end portion of the prosthetic heart valve towards an outflow end portion of the prosthetic heart valve, wherein the annular frame forms a central flow orifice extending from the inflow end portion to the outflow end portion and through the annular frame;
a plurality of commissure supports circumferentially disposed around the annular frame and projecting towards the outflow end portion; and
a plurality of leaflets secured to the plurality of commissure supports, wherein the plurality of leaflets are dehydrated and sterilized such that the prosthetic heart valve is configured to be stored in a dry environment, and wherein the plurality of leaflets are shape-set to a desired configuration after the plurality of leaflets are dehydrated and sterilized.

Example 47. The prosthetic heart valve of any of the above examples, wherein the plurality of leaflets are biased to an open configuration in which the plurality of leaflets are configured to permit blood flow through the central flow orifice.

Example 48. The prosthetic heart valve of any of the above examples, wherein the plurality of leaflets are biased to a closed configuration in which the plurality of leaflets are configured to occlude blood flow through the central flow orifice.

Example 49. The prosthetic heart valve of any of the above examples, wherein the plurality of leaflets are dehydrated by an exposure to a polyhydric alcohol and a C₁-C₃ alcohol.

Example 50. The prosthetic heart valve of any of the above examples, wherein the plurality of leaflets are sterilized by an exposure to an ethylene oxide gas.

Example 51. The prosthetic heart valve of any of the above examples, wherein the plurality of leaflets are shape-set when the plurality of leaflets are sterilized.

Example 52. A prosthetic heart valve comprising:
an annular frame extending from an inflow end portion of the prosthetic heart valve towards an outflow end portion of the prosthetic heart valve, wherein the annular frame forms a central flow orifice extending from the inflow end portion to the outflow end portion and through the annular frame;
a plurality of commissure supports circumferentially disposed around the annular frame and projecting towards the outflow end portion; and
a plurality of leaflets formed from bovine pericardium tissue, wherein:
   each of the plurality of leaflets comprises a free edge portion between two lateral portions,
   the two lateral portions are secured to adjacent ones of the plurality of commissure supports,
   each of the plurality of leaflets is biased to a desired configuration, and
   the prosthetic heart valve is configured to be stored in a dry state.

Example 53. The prosthetic heart valve of any of the above examples, wherein the prosthetic heart valve is configured for implantation at a mitral valve site.

Example 54. The prosthetic heart valve of any of the above examples, wherein the plurality of leaflets are biased to an open configuration in which the plurality of leaflets are configured to permit blood flow through the central flow orifice.

Example 55. The prosthetic heart valve of any of the above examples, wherein the free edge portion of each of the plurality of leaflets forms a generally bell-shaped curve in the open configuration, wherein the tip of the bell-shaped curve extends outwards towards a circumferential portion of the annular frame and each end portion of the bell-shaped curve terminates at one of the commissure posts adjacent the respective one of the plurality of leaflets.

Example 56. The prosthetic heart valve of any of the above examples, wherein the prosthetic heart valve is configured for implantation at an aortic valve site.

Example 57. The prosthetic heart valve of any of the above examples, wherein the plurality of leaflets are biased to a closed configuration.

Example 58. The prosthetic heart valve of any of the above examples, wherein the plurality of leaflets extend into a central portion of the central flow orifice to substantially occlude blood flow through the central flow orifice.

Example 59. The prosthetic heart valve of any of the above examples, wherein the plurality of leaflets are biased to an intermediate configuration, wherein each of the plurality of leaflets comprises a free edge portion, and wherein the free edge portions are partially extended towards the central flow orifice to partially occlude blood flow through the central flow orifice.

Example 60. The prosthetic heart valve of any of the above examples, wherein the prosthetic heart valve further comprises an outer skirt secured to an outer surface of the annular frame.

Example 61. The prosthetic heart valve of any of the above examples, wherein the prosthetic heart valve further comprises an inner skirt secured to an inner surface of the annular frame.

Example 62. The prosthetic heart valve of any of the above examples, wherein each of the plurality of leaflets comprises an inflow edge, and wherein each of the inflow edges is sutured to the inner skirt.

1. A packaging assembly for storing a prosthetic heart valve, the packaging assembly comprising:
a valve tray comprising an annular rim and a cavity extending away from the annular rim, wherein the cavity is configured to accept the prosthetic heart valve;
a lid configured to cover the annular rim; and
a leaflet biasing element disposed on a central portion of the lid, wherein:
the leaflet biasing element comprises an extrusion with a paraboloid shape,
the leaflet biasing element extends from a lower surface of the lid towards the valve tray, and
the leaflet biasing element is configured to extend at least partially into an inflow end portion of the prosthetic heart valve and secure a plurality of leaflets of the prosthetic heart valve in a desired configuration when the prosthetic heart valve is inserted in the cavity and the lid is closed over the annular rim.

2. The packaging assembly of claim 1, wherein the leaflet biasing element is configured to secure the plurality of leaflets of the prosthetic heart valve in an open configuration.

3. The packaging assembly of claim 2, wherein the leaflet biasing element is configured to extend at least partially through a central flow orifice of the prosthetic heart valve to contact the plurality of leaflets.

4. The packaging assembly of claim 1, wherein the valve tray further comprises a plurality of lower gas channels circumferentially disposed on the annular rim and projecting in a radial direction, wherein the plurality of lower gas channels are configured to permit gas flow into the cavity.

5. The packaging assembly of claim 4, wherein the lid further comprises a plurality of upper gas channels circumferentially disposed on the lid and projecting in a radial direction.

6. The packaging assembly of claim 5, wherein each of the upper gas channels is configured to align with a respective one of the lower gas channels when the lid is closed over the annular rim.

7. The packaging assembly of claim 1, wherein the packaging assembly further comprises a plurality of spacers circumferentially disposed on an inner lateral surface of the cavity.

8. The packaging assembly of claim 1, wherein the packaging assembly further comprises a circular boss extruded from the lid.

9. The packaging assembly of claim 8, wherein the circular boss is configured to frictionally engage an inner surface of the cavity between the annular rim and a lower ledge defined by and disposed beneath the annular rim.

10. A method of shape-setting a plurality of leaflets of a prosthetic heart valve to a desired configuration, comprising:
dehydrating the plurality of leaflets;
inserting the prosthetic heart valve into a packaging member, wherein the package member comprises:
a cavity configured to accept the prosthetic heart valve;
a lid configured to seal the cavity; and
a leaflet biasing element disposed on a central portion of a bottom surface of the lid, wherein the leaflet biasing element is configured to extend into the cavity, and
wherein the leaflet biasing element is configured to secure the plurality of leaflets in the desired configuration;
closing the lid such that the leaflet biasing element engages the plurality of leaflets; and
sterilizing the prosthetic heart valve.

11. The method of claim 10, wherein the prosthetic heart valve is dehydrated by exposing the prosthetic heart valve to a glycerol/ethanol mixture.

12. The method of claim 10, wherein the prosthetic heart valve is sterilized by exposing the prosthetic heart valve to an ethylene oxide gas.

13. The method of claim 10, wherein the leaflet biasing element is configured to engage the plurality of leaflets in an open configuration.

14. The method of claim 10, wherein the plurality of leaflets are secured in a closed configuration.

15. The method of claim 10, wherein the leaflet biasing element is configured to align with a central flow orifice of the prosthetic heart valve.

16. The method of claim 10, wherein the leaflet biasing element is configured to partially extend into a central flow orifice of the prosthetic heart valve when the lid is closed.
